(19) United States
(12) Patent Application Publication
(10) Pub. No.: US 2018/0078228 A1
(43) Pub. Date: Mar. 22, 2018

(54) METHOD AND MAGNETIC RESONANCE APPARATUS FOR AUTOMATIC SELECTION OF A SCAN PROGRAM FOR A MAGNETIC RESONANCE EXAMINATION OF A PATIENT

(71) Applicant: Siemens Healthcare GmbH, Erlangen (DE)

(72) Inventors: Alexander Janzik, Leinburg (DE); Lea Schubart, Erlangen (DE); Jens Thoene, Lauf an de Pegnitz (DE); Katharina Aschoff, Erlangen (DE); Kirstin Jattke, Nuernberg (DE); Mathias Nittka, Baiersdorf (DE)

(73) Assignee: Siemens Healthcare GmbH, Erlangen (DE)

(21) Appl. No.: 15/712,816

(22) Filed: Sep. 22, 2017

(30) Foreign Application Priority Data
Sep. 22, 2016 (DE) 102016218201.3

(51) Int. Cl. A61B 6/00; A61B 5/055; A61B 5/00; A61B 6/03; G01R 33/48

(52) U.S. Cl. CPC A61B 6/488; A61B 5/055; G06T 2207/30004; A61B 6/03; G01R 33/48; A61B 5/7271

(57) ABSTRACT

In a method and magnetic resonance (MR) apparatus for automatic selection of at least one scan program for a MR examination of a patient, at least one item of patient-specific information and/or at least one item of examination-specific information for the examination is provided as an input to a computer. Multiple available scan programs are made accessible to the computer. The computer is programmed to execute a self-learning algorithm, with the at least one item of information as an input to the self-learning algorithm, in order to automatically select the at least one scan program for the magnetic resonance examination from among the set of available scan programs. The computer then makes an electronic representation or designation of the at least one scan program that was the result of executing the self-learning algorithm available as an output from the computer.

### BACKGROUND OF THE INVENTION

### Field of the Invention

The present invention concerns a method for automatic selection of at least one scan program for a magnetic resonance examination of a patient using a magnetic resonance apparatus. Furthermore, the present invention concerns a magnetic resonance apparatus having a control computer that is designed to execute such a method for automatic selection of at least one scan program for a magnetic resonance examination of a patient using the magnetic resonance apparatus.

### Description of the Prior Art

For a magnetic resonance examination of a patient, it is currently necessary for the operating personnel supervising the magnetic resonance examination to search through a large number of available scan programs in order to find the appropriate and/or suitable scan program for the imminent magnetic resonance examination. For non-expert and/or inexperienced operating personnel, it is difficult and very time-consuming to select the appropriate and/or suitable scan program from among this large number of available scan programs.

### SUMMARY OF THE INVENTION

An object of the present invention is to provide a simple way of selecting a scan program for a magnetic resonance examination of a patient.

The method according to the invention for automatic selection of at least one scan program for a magnetic resonance examination of a patient using a magnetic resonance apparatus has the following steps.

At least one item of patient-specific information and/or at least one item of examination-specific information for the magnetic resonance examination is provided as an input to a computer.

The computer is programmed to execute a self-learning algorithm, with the at least one item of examination-specific information and/or the at least one item of patient-specific information as an input to the self-learning algorithm, in order to automatically select the at least one scan program for the magnetic resonance examination from among the set of available scan programs.

The computer then makes an electronic representation or designation of the at least one scan program that was the result of executing the self-learning algorithm available as an output from the computer.

In this context, a scan program means a set of control parameters, on the basis of which the magnetic resonance apparatus (or the scanner thereof) can be controlled, by a control computer, so image data can be generated. The image data generated using the control parameters of a scan program preferably have a uniform contrast and preferably represent a region of the examination object. In magnetic resonance examinations, a region of the examination object is often additionally represented in at least one further contrast. A further scan program having other control parameters can be used for this purpose. The magnetic resonance apparatus can therefore be controlled by multiple scan programs for the examination of the examination object.

For the control of the magnetic resonance apparatus, the control parameters of the selected scan programs are passed to the control computer of the magnetic resonance apparatus, with said control computer then controlling at least one apparatus component in accordance with the control parameters. If a control parameter is not a physical variable, the control computer can preferably translate the control parameter into at least one physical variable.

In order to use the magnetic resonance apparatus for magnetic resonance scanning, the magnetic resonance apparatus, and/or components incorporated in the magnetic resonance apparatus, are typically controlled with different control parameters that are available for selection depending on the magnetic resonance apparatus. The control parameters selected may depend on the examination object and/or on the clinical question. Control parameters can be physical variables, such as a voltage to be applied to a component, for example a radio-frequency antenna. A control parameter can also be a duration between two radio-frequency pulses and/or magnetic field gradients to be activated. By stipulating a resolution, for example, control parameters can characterize the magnetic resonance image data to be recorded.

The patient-specific information can be, for example, a height of the patient and/or an age of the patient and/or a gender of the patient and/or a weight of the patient and/or an etiopathology of the patient and/or a body region of the patient to be examined and/or further information that appears expedient to those skilled in the art. Patient-specific information can be provided manually by the operator, or read out automatically from a patient database by the control computer.

The examination-specific information can be an item of accessory information, for example. Furthermore, the examination-specific information can be an examination criterion. Examination-specific information can be provided manually by the operator, or provided automatically by the control computer.

The multiple available scan programs are preferably stored in a database. The database can be incorporated in the magnetic resonance apparatus. The database can also be embodied separately from the magnetic resonance apparatus, with data access from the magnetic resonance apparatus, for example via the control computer of, to the database being achieved by a data network. The database can also be made available centrally to multiple medical imaging apparatuses, in particular multiple magnetic resonance apparatuses that are each able to access the database via the data network.

Providing the multiple available scan programs includes making the scan programs available for the imminent magnetic resonance examination and able to be executed when a corresponding selection is made. The control computer of the magnetic resonance apparatus, preferably is the computer that executes the self-learning algorithm. The self-learning algorithm is typically based on machine learning, in which knowledge is generated from experience. The machine learning preferably takes place using artificial neural networks. Machine learning enables the self-learning algorithm to identify patterns and relationships in the learning data. Here the self-learning algorithm learns from these examples and generalizes these after the learning phase is complete.

In the automatic selection by execution of the self-learning algorithm, the self-learning algorithm can identify, for example on the basis of previous selections of scan programs, a pattern in the selection and/or in an association of scan programs with selection criteria, and the selection can be made on the basis of this pattern. In addition, the self-learning algorithm can make the selection on the basis of a frequency with which previous selections of scan programs were made.

The operator is preferably notified of the result of the automatic selection, for example by a graphical representation on a display monitor of the magnetic resonance apparatus. The notification of the automatic selection typically takes the form of a suggestion to the user. A final determination regarding which scan program will be executed is made by the user, in particular a member of medical operating personnel supervising the magnetic resonance examination. The scan program determined for the magnetic resonance examination is preferably stored in the database by the control computer, together with the at least one item of examination-specific information and/or the at least one item of patient-specific information, in order to be available for future selections of scan programs.

The invention enables a simple and time-saving selection of at least one scan program for the magnetic resonance examination on the patient. The automatic selection of the at least one scan program can relieve a user, in particular the medical operating personnel supervising the magnetic resonance examination, from having to carry out a laborious search through the available scan programs. A further advantage is that manual errors in the selection of the scan program can be prevented by the automatic selection of the at least one scan program. The self-learning algorithm also enables a continuously improving selection of a scan program to be made available, particularly if a subsequent determination by the user regarding the scan program is made available to the self-learning algorithm for future selections of scan programs.

In an embodiment of the invention, the multiple available scan programs are stored in the database together with at least one selection criterion. Here the selection criterion can be the examination-specific information and/or the patient-specific information, such as a body region on a patient to be examined, and/or further information that appears expedient to those skilled in the art. The selection criterion is preferably associated with the scan program by a scan program selection that has already been completed, as a function of the selection criterion for an earlier or preceding magnetic resonance examination. On the basis of the available selection criterion the scan programs can be searched for a correlation with the provided patient-specific information and/or the examination-specific information provided, and the selection criteria. This allows a rapid, simple association of at least one scan program among the multiple available scan programs, with the examination-specific information and/or the patient-specific information.

In another embodiment of the invention, the at least one item of examination-specific information is an item of accessory information. The accessory information can be an item of information about an available and/or required accessory unit, such as a local radio-frequency coil unit for acquiring magnetic resonance signals and/or an injection unit for administering a contrast medium and/or an ECG unit, etc., with the accessory unit being used for the imminent magnetic resonance examination on the patient. The accessory information can be entered manually by the medical operating personnel, or determined autonomously and/or automatically by the control computer. Plug signals from a plugged-in plug of a radio-frequency coil, or from a plugged-in plug of an ECG unit, for example, may be made available to the control computer for the automatic determination of the accessory information, with the control computer being able to generate corresponding accessory information on the basis of the plug signals. This embodiment of the invention allows the automatic selection of the at least one scan program to be matched to the imminent magnetic resonance examination, and a corresponding scan program to be selected that is compatible with the accessory unit that is available and/or to be used.

Alternatively or in addition, the at least one item of examination-specific information can be an examination criterion. The examination criterion can comprise, for example, an examination time and/or an image resolution and/or a contrast, etc. This also allows the automatic selection of the at least one scan program to be matched to the imminent magnetic resonance examination and a corresponding scan program to be selected that is compatible with the examination criterion.

In another embodiment of the invention, in the method step of selecting the at least one scan program, multiple scan programs for the magnetic resonance examination are selected from among the set of available scan programs, with the multiple selected scan programs being evaluated for a correlation with the at least one item of examination-specific information and/or the at least one item of patient-specific information. The evaluation preferably identifies a level of correlation of the selected scan program in each case with the at least one item of examination-specific information and/or the at least one item of patient-specific information. This also enables non-expert users (non-expert and/or inexperienced medical operating personnel) to easily assess and/or evaluate the selection of selected scan programs obtained.

In a further embodiment of the invention, the scan program with the highest correlation in the evaluation is represented graphically as a suggestion to the user. The suggestion is preferably displayed or shown to a user, in particular the medical operating personnel supervising the magnetic resonance examination, at a display monitor of a user console and/or a user interface. This has the advantage that inexperienced users can determine, on the basis of the suggestion, and in an easy and time-saving manner, a suitable scan program for the imminent magnetic resonance examination on the patient. A laborious search through the available scan programs by the user to find an appropriate and/or suitable scan program is then not necessary.

In another embodiment of the invention, the scan program determined for the magnetic resonance examination is stored in a database together with the at least one item of examination-specific information and/or the at least one item of patient-specific information. The scan program is preferably stored together with the at least one item of examination-specific information and/or the at least one item of patient-specific information in the same database that already has the multiple available scan programs stored therein. The storage and/or saving of the scan program together with the at least one item of examination-specific information and/or the at least one item of patient-specific information is preferably done by the control computer of the magnetic resonance apparatus. This embodiment of the invention has the advantage that a continuously improving selection of a scan program can be made available, and consequently a quality of the acquired image data can be improved.

Furthermore, the invention includes a magnetic resonance apparatus with a control computer and a database, wherein the control computer is configured to execute the inventive method for automatic selection of at least one scan program for a magnetic resonance examination of a patient.

The magnetic resonance device designed in this way allows for a simple and time-saving selection of at least one scan program for the magnetic resonance examination on the patient. The automatic selection of the at least one scan program relieves a user, in particular the medical operating personnel supervising the magnetic resonance examination, having to carry out a laborious search through the available scan programs. A further advantage is that manual errors in the selection of the scan program can be prevented by the automatic selection of the at least one scan program. The self-learning algorithm also enables a continuously improving selection of a scan program to be made available, particularly if a subsequent determination by the user regarding the scan program is made available to the self-learning algorithm for future selections of scan programs.

The advantages of the magnetic resonance apparatus essentially correspond to the advantages of the inventive method for an automatic selection of at least one scan program for a magnetic resonance examination of a patient, as explained above in detail. Features, advantages and alternative embodiments mentioned above are also applicable to the apparatus.

The present invention also encompasses a non-transitory, computer-readable data storage medium encoded with programming instructions (program code) that, when the storage medium is loaded into a computer or computer system of a magnetic resonance apparatus, cause the computer or computer system to operate the magnetic resonance apparatus in order to implement any or all of the embodiments of the inventive method, as described above.

### BRIEF DESCRIPTION OF THE DRAWINGS

FIG. 1 is a schematic illustration of a magnetic resonance apparatus according to the invention.

FIG. 2 is a flowchart of the inventive method for automatic selection of at least one scan program for a magnetic resonance examination of a patient.

### DESCRIPTION OF THE PREFERRED EMBODIMENTS

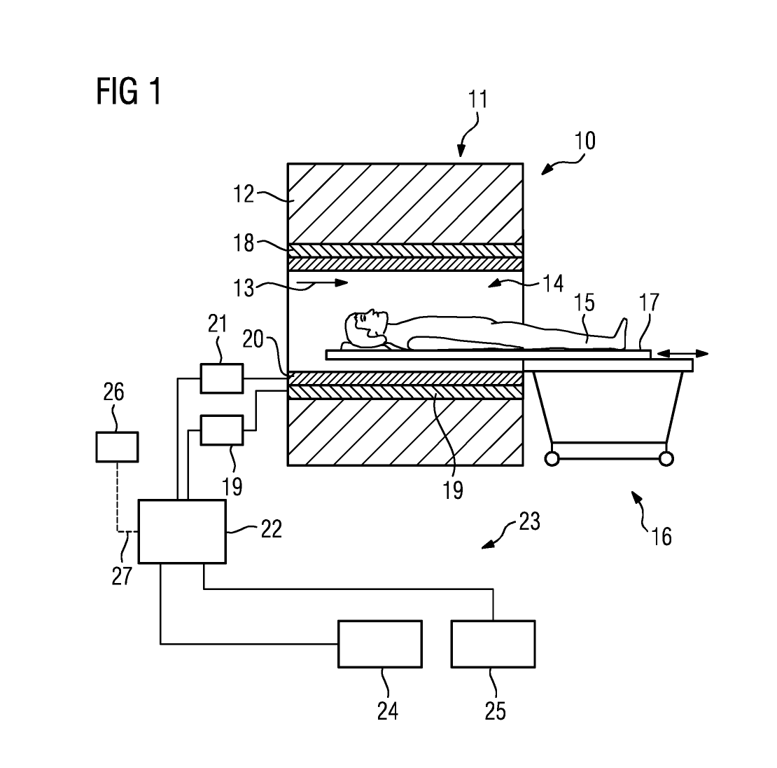

FIG. 1 is a schematic illustration of a magnetic resonance apparatus 10. The magnetic resonance apparatus 10 has a scanner 11 that has a superconducting basic field magnet 12 that generates a strong and constant basic magnetic field 13. In addition, the scanner 11 has a patient accommodating region 14 to receive a patient 15. In the exemplary embodiment, the patient accommodating region 14 is configured to be cylindrical and is peripherally surrounded cylindrically by the scanner 11. In principle, however, a configuration of the patient accommodating region 14 deviating therefrom is possible. The patient 15 can be moved by a patient support 16 into the patient accommodating region 14. For this purpose, the patient support 16 has a patient table 17 that is movable within the patient accommodating region 14.

The scanner 11 also has a gradient coil arrangement 18 that generates magnetic field gradients that are used for spatially encoding the MR signals during an imaging process. The gradient coil arrangement 18 is controlled by a gradient controller 19. The scanner 11 furthermore has a radio-frequency (RF) antenna 20 an RF controller 21 so as to radiate RF magnetic resonance sequences into an examination space that is substantially formed by the patient accommodating region 14 of the scanner 11. The radiated RF sequence gives certain nuclear spins in the patient 15 a magnetization that causes those nuclear spins to deviate from the basic magnetic field 13. As the excited nuclear spins relax and return to the steady state, they emit the aforementioned MR signals (also RF signals) which are detected by the same antenna from which the RF sequence was radiated, or by a different antenna.

For controlling the basic field magnet 12, the gradient controller 19, and the RF controller 21, the magnetic resonance apparatus 10 has a control computer 22. The control computer 22 centrally controls the scanner 11, such as to perform a predetermined imaging gradient echo sequence. Furthermore, the control computer 22 has an evaluation processor (not shown) for evaluating medical image data acquired during the magnetic resonance examination. Furthermore, the magnetic resonance apparatus 10 has a user interface 23, which is connected to the control computer 22. Control information such as imaging parameters and reconstructed magnetic resonance images can be displayed on a display unit 24, for example on at least one monitor, of the user interface 23 for medical operating personnel. In addition, the user interface 23 has an input unit 25 via which information and/or parameters can be input by the medical operating personnel during a scanning procedure.

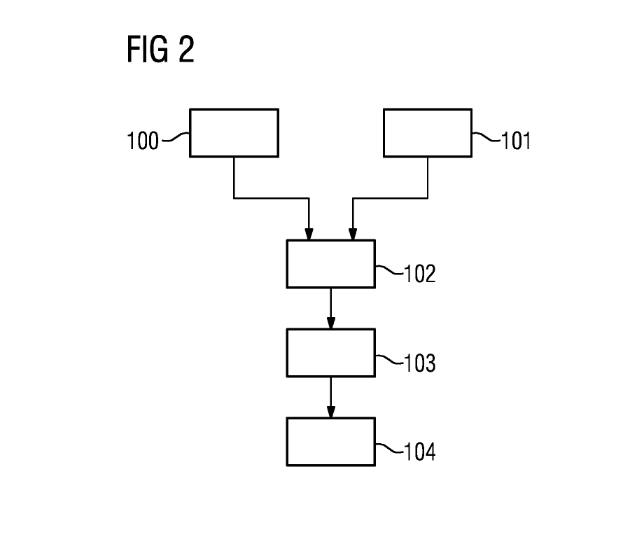

FIG. 2 shows the inventive method for an automatic selection of at least one scan program for a magnetic resonance examination of a patient 15 using the magnetic resonance apparatus 10. The control computer 22 of the magnetic resonance apparatus 10 is designed for the execution of the method. For this purpose, the control computer 22 has computer programs and/or software, which can be loaded directly in a storage unit, with program code in order to execute the method for setting and/or adjusting scan parameters for a scan sequence of a magnetic resonance examination when the computer programs and/or software is run in the control computer 22. For this purpose, the control computer 22 has a core processor (not shown) that is designed to run the computer programs and/or software, and has the storage unit in which the software and/or computer programs are saved and/or stored.

The software and/or computer programs can be stored on an electronically readable data storage medium that is embodied separately from the control computer 22 and/or separately from the magnetic resonance apparatus 10. The electronically readable data storage medium can be loaded into the control computer 22.

In a first method step 100 of the method, at least one item of patient-specific information and/or at least one item of examination-specific information for the magnetic resonance examination is/are entered into the control computer 22. The at least one item of patient-specific information can be, for example, a height of the patient 15 and/or an age of the patient 15 and/or a gender of the patient 15 and/or a weight of the patient 15 and/or an etiopathology of the patient 15 and/or a body region of the patient 15 to be examined and/or further information that appears expedient to those skilled in the art.

The at least one item of patient-specific information can be entered manually by a user, in particular the medical operating personnel supervising the magnetic resonance examination on the patient 15, via the user interface 23, in particular the input unit 25 of the user interface 23. The at least one item of patient-specific information can also be read out automatically from a patient database by means of the control computer 22 of the magnetic resonance apparatus 10.

The examination-specific information can be an item of accessory information, i.e. an item of information about an accessory unit that is available and/or required for the imminent magnetic resonance examination on the patient 15. The accessory information can be a local radio-frequency coil unit for acquiring magnetic resonance signals and/or an injection unit for administering a contrast medium and/or an ECG unit, etc. Here the accessory information can be entered manually by the medical operating personnel, or determined autonomously and/or automatically by the control computer 22. Plug signals from a plugged-in plug of a radio-frequency coil, or from a plugged-in plug of an ECG unit, for example, may be made available to the control computer 22 for the automatic determination of the accessory information, with the control computer 22 being able to generate corresponding accessory information on the basis of the plug signals.

Alternatively or in addition, the at least one item of examination-specific information can be an examination criterion. The examination criterion can be, for example, an examination time and/or an image resolution and/or a contrast, etc.

In a further method step 101 multiple available scan programs are provided. The scan programs are stored in a database 26, with the control computer 22 being able to access the database 26 by means of a data network 27. The method step 101 of providing the multiple available scan programs and the method step 100 of providing the at least one item of patient-specific information and/or the at least one item of examination-specific information for the magnetic resonance examination can take place simultaneously, or in any sequence.

The multiple available scan programs are preferably saved and/or stored with at least one selection criterion in the database 26. The selection criterion is associated with the scan program by a scan program selection that has already been completed, as a function of the selection criterion for an earlier or preceding magnetic resonance examination. Here the selection criterion can be the examination-specific information and/or also the patient-specific information, such as a body region on a patient 15 to be examined, for example, and/or further information that appears expedient to those skilled in the art.

In a method step 102 that follows the method step 101 of providing the multiple available scan programs and the method step 100 of providing the at least one item of patient-specific information and/or the at least one item of examination-specific information for the magnetic resonance examination, the at least one scan program is automatically selected for the magnetic resonance examination from among the set of available scan programs on the basis of the at least one item of examination-specific information and/or the at least one item of patient-specific information. For this purpose the control computer 22 has a self-learning algorithm by which the automatic selection is made.

The self-learning algorithm searches through the database 26 with the available scan programs, for example, and compares the selection criteria stored with the scan programs for a correlation with the patient-specific information provided and/or the examination-specific information provided. In this case, in the method step 102 of automatically selecting the at least one scan program, multiple scan programs for the imminent magnetic resonance examination can also be selected by the self-learning algorithm from among the set of available scan programs. The selection of the scan program or also the selection of the multiple scan programs is/are displayed for the user in a further method step 103, so that the user can determine, on the basis of the selected scan programs, at least one scan program for the magnetic resonance examination on the patient 15. The selection is displayed at the display unit 24 of the user interface 23.

Here the selected scan programs are also evaluated by the control computer 22 with regard to a level of a correlation of the selected scan program in each case with the at least one item of examination-specific information and/or the at least one item of patient-specific information. In addition, the control computer 22 marks for the user whichever scan program has the greatest correlation between the selection criteria for the scan program and the patient-specific information provided and/or the examination-specific information provided. Furthermore, the suggestion is also represented graphically for the user at the display unit 24 of the user interface 23.

The scan program marked as a suggestion can be marked for example with color coding. In addition, the scan program marked as a suggestion can also appear first on a selection list that has all selected scan programs. In addition, other markings of the scan program marked as a suggestion, which appear expedient to those skilled in the art, are also conceivable.

On the basis of the selection of the scan program or the selection of multiple scan programs and the suggestion marked graphically, the user can determine at least one scan program that will be used to perform the magnetic resonance examination on the patient 15. In a further method step 104, the scan program determined for the magnetic resonance examination, together with the at least one item of examination-specific information and/or the at least one item of patient-specific information, is saved and/or stored in the database 26 by the control computer 22. The at least one item of examination-specific information and/or the at least one item of patient-specific information, is then saved and/or stored in the database 26 as a selection criterion for the scan program.

Although modifications and changes may be suggested by those skilled in the art, it is the intention of the Applicant to embody within the patent warranted hereon all changes and modifications as reasonably and properly come within the scope of the Applicant's contribution to the art.

1. A method for automatic selection of at least one scan program for a magnetic resonance (MR) examination of a patient using an MR data acquisition scanner, said method comprising:
providing a computer with at least one item of information selected from the group consisting of patient-specific information and examination-specific information, for said MR examination;
providing said computer with access to multiple, available scan programs;
in said computer, executing a self-learning algorithm, with said at least one item of information as an input to said algorithm, in order to automatically select at least one scan program, from among said multiple, available scan programs, for executing said MR examination, thereby producing at least one selected scan program; and
making an electronic representation of said at least one selected scan program available from the computer as an electronic output.

2. A method as claimed in claim 1 comprising storing said multiple, available scan programs in a database, that is accessible by said computer, together with at least one selection criterion also stored in said database respectively with said scan programs.

3. A method as claimed in claim 1 wherein said at least one item of examination specific information comprises an item of accessory information for an accessory to be used with said MR examination.

4. A method as claimed in claim 1 wherein said at least one item of examination specific information comprises an examination criterion for said MR examination.

5. A method as claimed in claim 1 comprising executing said self-learning algorithm in order to produce multiple scan programs as an output of said self-learning algorithm, and, in said computer, evaluating each of the multiple, selected scan programs with regard to a correlation with said at least one item of information.

6. A method as claimed in claim 5 comprising providing a scan program, among said multiple, selected scan programs, having a highest correlation in said evaluation with said at least one item of information, and presenting said scan program with the highest correlation at a graphical display as a suggestion to a user of the magnetic resonance data acquisition scanner.

7. A method as claimed in claim 5 comprising storing the scan program with the highest correlation in a database together with said at least one item of information.

8. A magnetic resonance (MR) apparatus comprising:
an MR data acquisition scanner;
a computer provided with at least one item of information selected from the group consisting of patient-specific information and examination-specific information, for said MR examination;
said computer having access to multiple, available scan programs;
said computer being configured to execute a self-learning algorithm, with said at least one item of information as an input to said algorithm, in order to automatically select at least one scan program, from among said multiple, available scan programs, for executing said MR examination, thereby producing at least one selected scan program; and
said computer being configured to make an electronic representation of said at least one selected scan program available from the computer as an electronic output.

9. A non-transitory, computer-readable data storage medium encoded with programming instructions, said storage medium being loaded into a computer system of a magnetic resonance (MR) apparatus that comprises an MR data acquisition scanner, said programming instructions causing said computer system to:
receive at least one item of information selected from the group consisting of patient-specific information and examination-specific information, for said MR examination;
access multiple, available scan programs;
execute a self-learning algorithm, with said at least one item of information as an input to said algorithm, in order to automatically select at least one scan program, from among said multiple, available scan programs, for executing said MR examination, thereby producing at least one selected scan program; and
make an electronic representation of said at least one selected scan program available from the computer system as an electronic output.
